(19) United States
(12) Patent Application Publication
(10) Pub. No.: US 2022/0249278 A1
(43) Pub. Date: Aug. 11, 2022

(54) Therapeutic Joint Compression, Comfort, Cooling, and Heating Compress Pad and Wrap

(71) Applicants: Keith Ablow, Newburyport, MA (US); Kathi Fairbend, Weston, MA (US)

(72) Inventors: Keith Ablow, Newburyport, MA (US); Kathi Fairbend, Weston, MA (US)

(21) Appl. No.: 17/526,222

(22) Filed: Nov. 15, 2021

Related U.S. Application Data
(60) Provisional application No. 63/113,890, filed on Nov. 15, 2020.

(51) Int. Cl. A61F 7/02; A61F 13/06; A61F 13/10; A61F 13/14

(52) U.S. Cl. CPC A61F 7/02; A61F 13/061; A61F 2007/023; A61F 13/101; A61F 13/146; A61F 13/067

(57) ABSTRACT

A versatile therapeutic joint compression, comfort, cooling, and heating compress pad and wrap consisting of a dome shaped receptacle member designed to hold a hot or cold compress pack or with a gel insert that is neither heated nor cooled, in an interior compartment, as well as a crescent shaped compression flange member, the combination of the dome (cap) and flange (brim) forming a structure similar to a baseball cap, and additionally a plurality of flexible straps to affix or secure the cap and brim to a variety of human joints such as a knee, heel, elbow, or shoulder for treating a great variety of medical conditions relating thereto.

### CROSS REFERENCE TO RELATED APPLICATIONS

This application is based on the disclosure of U.S. Provisional Application No. 63/113,890 filed Nov. 15, 2020 by the same inventors which disclosure is herein incorporated by reference.

### BACKGROUND OF THE INVENTION

### Field of the Invention

This invention resides in the field of medical devices used to treat joint diseases and/or injuries using compression and hot and/or cold compress pads and wraps.

### Description of the Prior Art:

A substantial amount of material relating to devices designed to treat various medical conditions in joints of the human anatomy utilizing pads and wraps to apply heat, cooling, and compression to those joints exists in the prior art.

For example, U.S. Pat. No. 5,476,442 shows a hinged two-part assembly for heat application to a knee secured to a leg by VELCRO straps. Also U.S. Pat. No. 4,676,247 shows a wrap with compartments for gel packs and EP0733385 discloses permanently installed protective pads.

In contrast to these, the present invention employs a combination of structural elements arranged to provide therapeutic treatment for a great number of specific ailments to several different joints in either of two different up or down orientations as is disclosed below.

### SUMMARY OF THE INVENTION

The invention may be summarized as a versatile therapeutic joint compression, comfort, cooling, and heating compress pad and wrap consisting of a dome shaped receptacle member made of, for example, a flexible medical grade fabric, which dome is arranged to hold a hot or cold compress pack or with a gel insert that is neither heated nor cooled in an interior compartment, as well as a crescent shaped compression flange member attached to the base of the dome made of, for example, a semi-flexible plastic sheet sewn inside a covering of medical grade fabric, the combination of the dome (cap) and flange (brim) forming a structure similar to a baseball cap, and additionally, a means, for example, a plurality of flexible straps, to affix or secure the cap and brim to a variety of human joints such as a knee, heel, elbow, or shoulder.

The pad-wrap is arranged to fit different joints by adjusting the size of the bottom parameter or base of the dome using a combination of one or more cutouts and straps and can be oriented in either a flange (brim) up or flange down position to provide treatment options for a multitude of maladies as will be presented in the following description of the preferred embodiment.

Further features of the invention include:

The extended compression area afforded by the brim portion of the invention, the brim being short or long in various permutations of the device, compared to the quite minimal compression area afforded by the anchoring strap of the cap portion of the invention makes the design and function of the invention unique.

The invention is intended to offer comfort and protection and compression and hot or cold application to areas of the body for any discomfort whatsoever, but, and depending on the orientation of the brim, a unique feature of the invention, including overuse injuries to the patellofemoral joint, as well as mild to moderate degenerative changes, patella tendonitis, quadriceps tendonitis and tendinopathy, lateral tracking syndrome, bursitis, Osgood Schlatter's syndrome, etc.

The invention may be fashioned from comfortable, stretchy material that is infused with copper ions or silver ions for antimicrobial purposes or other known uses of copper or silver-infused fabrics. The material on the inside of the cap must be thin enough to transmit hot or cold from the hot or cold pack that fits inside the cap when that additional function, heating or cooling, is desired. The material of the brim will be of sufficient stiffness to transmit pressure to the selected joint as provided by the flexible straps which secure the invention to the joint. All materials will be composed of appropriate medical grade standards as required.

This pad-wrap may be made available in different sizes for different applications. Alternative sizing and slightly different contours and sizing of the cap and brim as well as the presence or absence of straps on either the cap or brim will allow, as stated above, the invention to be used on a knee, elbow, shoulder or heel.

The invention with a very extended brim can include attachment points for an over-the-opposite-shoulder strap accessory that turns the elbow version of the invention into a sling with unique properties of cradling the elbow and providing compression to the forearm.

The invention can be used on either side, right or left, of the body and made available in a range of child and adult sizes.

Also, the baseball cap appearance of the invention confers upon it a unique embellishment licensing potential via branding with major league baseball, minor league baseball, high school and college baseball or even Little League baseball team names or logos.

### DESCRIPTION OF THE DRAWINGS

FIG. 1 is a perspective view of a preferred embodiment of the invention;

FIG. 2 is a front view of the embodiment of FIG. 1;

FIG. 3 is a rear view of the embodiment of FIG. 1;

FIG. 4 is a right side elevation view of the embodiment of FIG. 1;

FIG. 5 is a left side elevation view of the embodiment of FIG. 1;

FIG. 6 is a top view of the embodiment of FIG. 1;

FIG. 7 is an bottom view of the embodiment of FIG. 1;

FIG. 8A is an illustration of the embodiment of FIG. 1 in use;

FIG. 8B is another illustration of the embodiment of FIG. 1 in use;

FIG. 9 is an additional illustration of the embodiment of FIG. 1 in use; and

FIG. 10 is a further illustration of the embodiment of FIG. 1 in use.

### DESCRIPTION OF THE PREFERRED EMBODIMENT

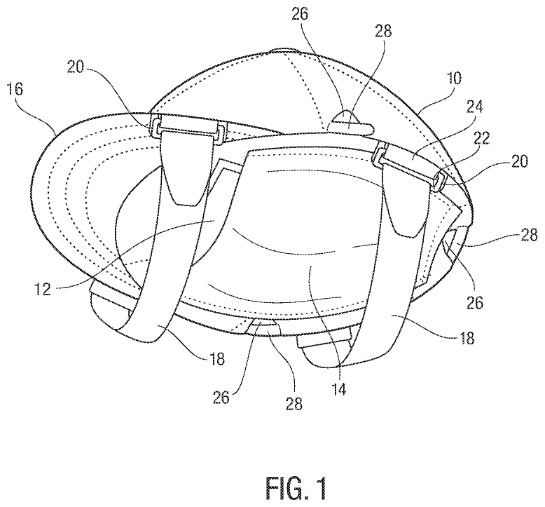

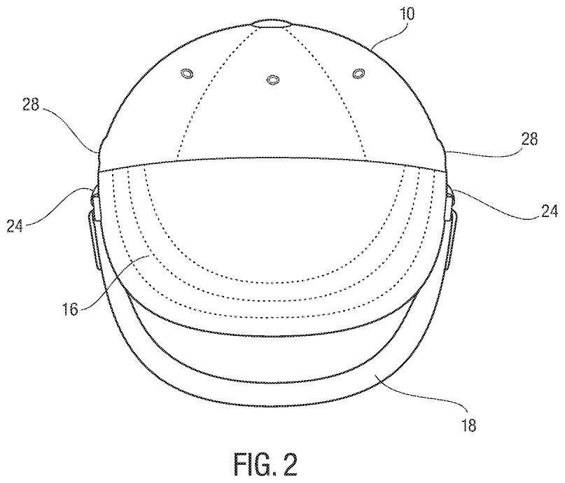

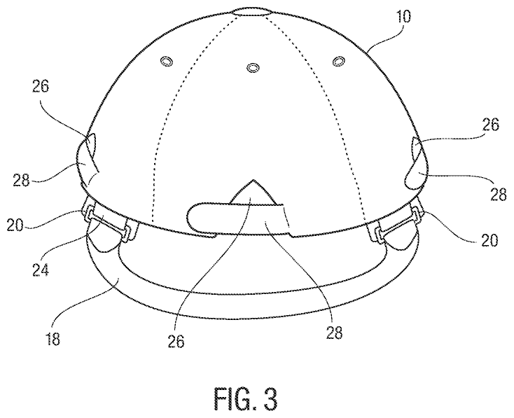

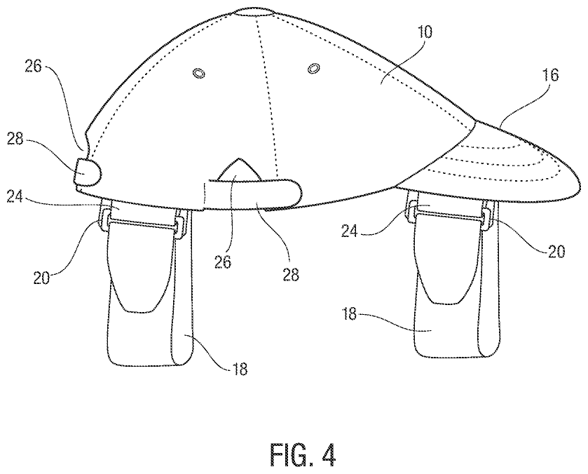

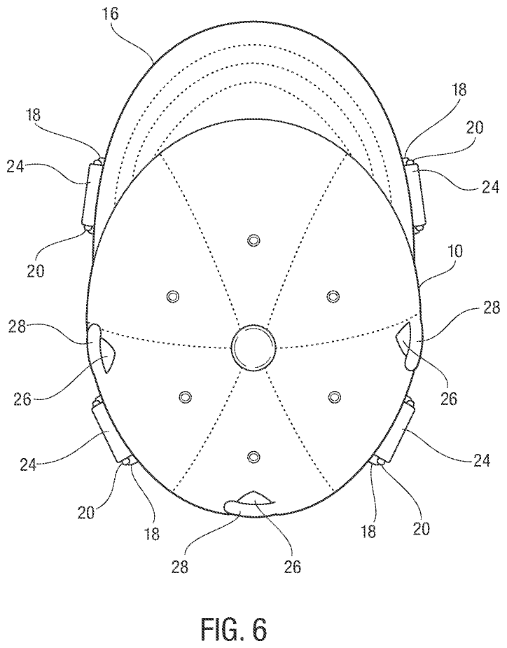

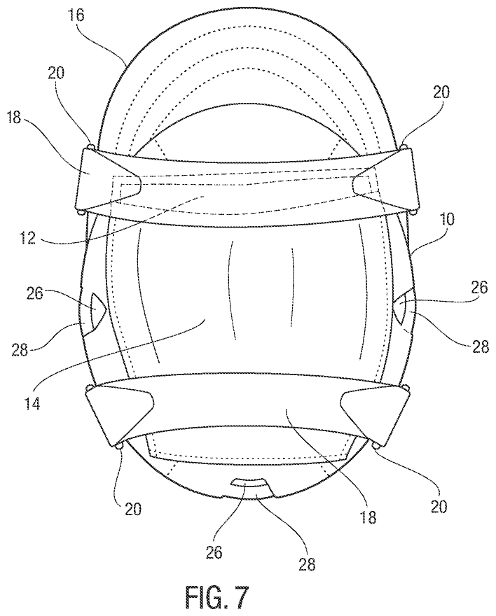

As is shown in the plurality of views of FIGS. 1-7, the preferred embodiment is composed of a padded, dome-shaped receptacle or cap 10 designed to hold a hot or cold compress pack 12 in an interior compartment 14 within the dome or alternatively, to hold a gel insert that is neither heated nor cooled, and to which dome is attached a crescent-shaped compression flange or brim 16 at the bottom parameter of the dome.

Means for affixing, or binding the pad-wrap, for example, a plurality of straps 18 composed of, for example, stretched VELCRO, attached to the cap and brim by strap connection assemblies 20, are employed to secure the cap and brim to a human joint such as a knee, heel, elbow, or shoulder. The exemplary connection assemblies are composed of a bridge bar 22, the top part of which is attached to the cap or brim by fabric loops 24 and the bottom providing a support and pass through for straps 18.

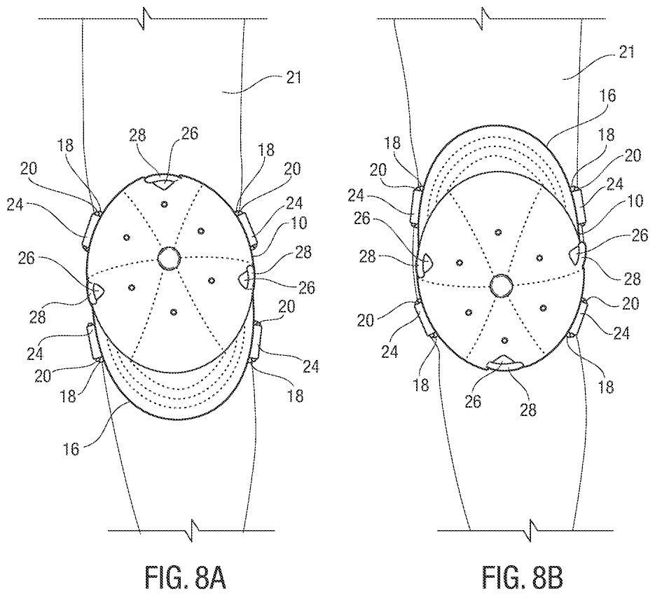

The strap holding the brim in place may be of any width including nearly as wide as the brim itself and will also provide compression to the area due to the function of the brim either directly above or directly below a joint, since the brim can be oriented either above or below the joint 21, for example, a knee on a leg as is shown in FIGS. 8A and 8B. These straps can also provide their own more minimal zone of compression by virtue of their ability to be tightened onto, in the case of a knee joint, the thigh or calf.

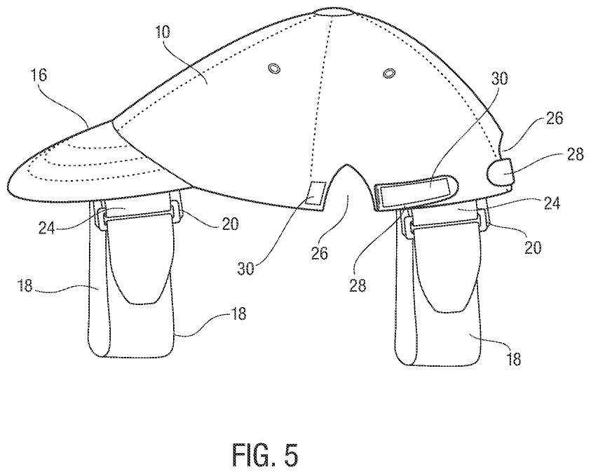

Additional versatility allowing the pad-wrap to fit a variety of joints and anatomical sizes is provided by a provision to adjust the size of the bottom periphery of the cap 10 using one or more cap cutouts 26 and tabs 28 employing, as shown in FIG. 5, mating VELCRO pads 30 to shrink or expand that portion of the unit.

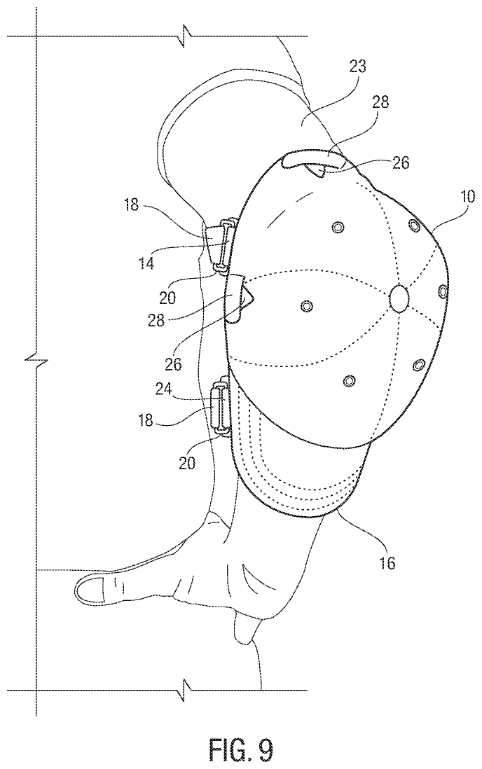

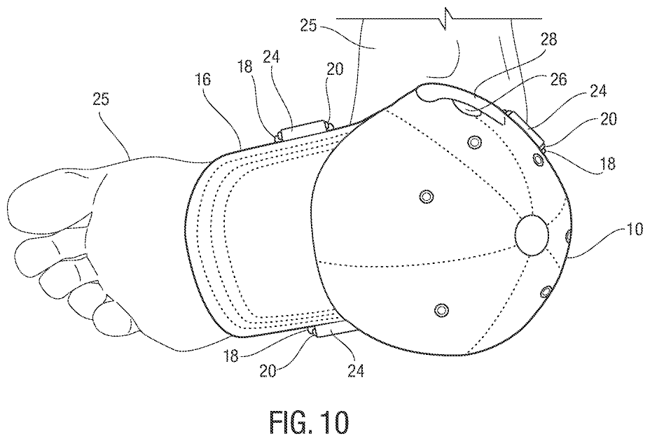

FIG. 9 shows the pad-wrap disposed on an elbow 23, and FIG. 10, on a heel 25.

The following is a list of positions or orientations of the invention, brim upwards or downwards in relation to the head of the body. as shown in FIGS. 8A and 8B, matched with particular joints and diseases and/or injuries.

Knee Conditions and/or Diagnoses that would Benefit from the Invention

Many varieties of overuse injuries, such as strains, sprains and bruises, frequently occur in the knee joint itself and surrounding muscles, tendons, and ligaments.

The first line of defense for musculoskeletal injuries includes ice, hence the moniker RICE (Rest, Ice, Compress, Elevate)

For injury to the patellomfemoral joint, referred to as Runner's Knee, the brim advantage would be in the Up position.

For Plica Syndrome of the patellomfemoral joint, the brim would also be Up.

For mild to moderate Degenerative Arthritis of the joint would also use the brim Up.

Quadriceps Tendonitus and Quadriceps Tendonopathy would also use the brim Up.

Lateral Tracking Syndrome would use the brim Up or Down.

Patella Tendonitus, Infrapateller Bursitis, Osgoods-Schlatters Disease, and Illiotibial Band Syndrome would call for the brim to be Down

Elbow Conditions and/or Diagnoses that would Benefit from the Invention

The elbow would be placed in the dome of the cap for each indication, as with the knee joint and the heel use of the invention.

The orientation of the brim and the invention itself is dependent on the anatomical markers and the problem. As the elbow joint is compact, elbow injuries often involve multiple structures. The Elbow is subjected to many overuse injuries.

### Diagnoses:

Lateral Epicondylitis (known as Tennis elbow). This occurs frequently from poor mechanics and overuse of the extensor muscles of muscles of wrist and forearm. Brim Up.

Medial Epicondylitis (known as Golfer's elbow). As with Lateral Epicondylitis this is an overuse syndrome from poor mechanics, in this instance from the flexor muscles of the hand wrist and forearm. Brim Up.

Cubital Tunnel Syndrome resulting in ulnar nerve irritation. Nerve is very superficial and located in a bony groove close to the surface. Brim Up.

Ulnar Collateral ligament can be strained and frequently has small tears due to impact and overuse. Brim Up or Down.

Radial collateral ligament can also be strained due to impact and overuse. Brim Up or Down.

Bony spurs and arthritis in the joint, usually caused by over use and use. Brim Up or Down.

Biceps tendonitis, strain or tear or even rupture at it's insertion on radius. Brim Up or Down.

Triceps tendonitis, strain or tear. Brim Up.

Olecranon bursitis, from sudden movement, over use of elbow, weight bearing on the elbow. Brim Up.

Heel Conditions and/or Diagnoses that would Benefit from the Invention

Heel placed in the dome of the cap for each indication, as with the knee use for the knee joint. This provides cushioning and protection and cooling or warming.

The orientation of the brim and the invention is dependent on the anatomical markers and location of the problem.

### Diagnoses:

Plantar fasciitis: Plantar fasciitis can involve not only the plantar surface of the foot but the attachment of the Achilles tendon on the posterior of the heel. Brim up toward the knee or down toward the toes.

Achilles tendonitis, tendonosis or tendonopathy: Achillles tendon originates from the calf, travels down to the calcaneus (heel) and then splays out at the heel and goes under the heel to attach to the plantar fascia. Brim up toward the knee or down toward the toes.

Peroneal tendonitis, tendonosis or tendonopathy: More than one peroneal tendon starts in the lower leg and travels under the foot. Brim up toward the knee or down toward the toes.

Heel spur: A localized problem of the tissues surrounding the heel. Brim should be position down toward the toes.

Calcaneal fat pad inflammation (medial, lateral and/or central): Brim should be positioned down toward the toes.

Sever's disease or syndrome: Inflammation of the growth plate at the back of the heel where the Achilles tendon attaches: Brim should be positioned up toward the knee.

Tarsal tunnel syndrome: The tarsal tunnel is the only effective way to enter the space where heel and ankle joint meet. It is important to ice or heat through the back of the ankle joint. The brim should be positioned up toward the knee.

Osteoarthritis, Traumatic or Systemic Arthritis, including gout, lupus and psoriatic arthritis: Brim should be positioned up toward the knee.

Calcaneal cysts: These are cysts within the heel bone. Brim should be positioned up toward the knee.

Retrocalcaneal bursitis: Inflammation causing pain in the back of the heel, often under the point of attachment of the Achilles tendon on the heel. Brim should be positioned up toward the knee.

Proximal metatarsal fracture: The brim should be positioned up toward the knee.

Metatarsalgia: Provided our incarnation includes a long brim, this can be addressed with the invention with the brim positioned down toward the toes.

### Shoulder Issues Addressed by the Invention

The shoulder joint would be positioned in the dome of the cap. The cap provides comfort, protection, cradling, cooling or warming. Unlike other joints we have discussed, The invention can be positioned in four ways: anteriorly, posteriorly and laterally when the brim is pointed toward the elbow and laterally when the brim is pointed toward the neck.

With the brim pointed toward the neck, the strap on the brim must be long enough to encircle the torso. Hence, this is a different embodiment of the design (length of strap must be far longer or retrofitted with an additional longer strap attached with Velcro to the brim).

As with all forms of the invention the anatomical markers and injury/discomfort determine the position in which the invention is best used.

The shoulder joint is part of the shoulder girdle complex, and its injuries require paying respect to the shoulder joint itself, the acromioclavicular joint, as well as the scapula (the shoulder blade, which lends support to the shoulder).

Rotator cuff Injuries: The rotator cuff surrounds the shoulder joint, stabilizes it and facilitates important and finely tuned movements. It maintains the head of the humerus securely in the shoulder socket and prevents subluxation or dislocation of the shoulder and subsequent impairment of motion and strength. Injuries to the rotator cuff vary in degree from muscle strain, to tendinitis, to microscopic tears of muscle or tendon, to full thickness tears of a muscle or tendon from its point of attachment.

When the brim is oriented pointing toward the elbow, the invention can be useful with the brim facing anteriorly (cradling, comforting and compressing the biceps), laterally (cradling, comforting and compressing a constellation of muscles, including the deltoid) or posteriorly (cradling, comforting and compressing the triceps). When the brim is pointed toward the neck, NEEDCAPS are used with the brim in the lateral position (cradling, comforting and compressing muscles including the surpraspinatus).

Acromioclavicular separation (also known as AC separation): The acromioclavicular joint joins the clavicle to the scapula. When there is an injury to that joint, the invention should be oriented with the brim toward the neck.

Acromioclavicular bursitis: the invention should be oriented with the brim pointing toward the neck.

Impingement syndrome: This is rotator cuff tendinitis in the subacromial space. the invention should be oriented with the brim pointing toward the neck.

Adhesive capsulitis: This is also known as frozen shoulder. the invention may be positioned with the brim pointing toward the neck or the elbow.

Arthritis affecting the shoulder joint: Brim pointed toward neck, surface of brim anterior, lateral or posterior.

Cartilage tears: These put the shoulder joint at risk of arthritis. the invention should be positioned with the brim pointing toward the neck.

Bony spurs of the shoulder joint: These result from overuse of the shoulder. the invention can be positioned with the brim pointing toward the neck or the elbow.

Tendonitis or rupture of the biceps tendon attachment at the shoulder joint: the invention can be positioned with the brim pointing toward the neck or the elbow.

Deltoid bursitis (at the attachment of the deltoid muscle on lateral side of the arm): the invention can be positioned with the brim pointing toward the elbow.

What is claimed is:

1. A versatile therapeutic joint compression, comfort, cooling, and heating compress pad and wrap comprising in combination:
A. a dome shaped receptacle member;
B. a pocket disposed within said dome, said pocket arranged to hold an insertable unit designed for treatment of a medical disease or injury of said joint;
C. a crescent shaped compression flange member attached to the lower periphery of said dome, the combination of said dome and said flange forming a structure similar to a baseball cap; and
D. a plurality of flexible straps attached to said dome and said flange to affix and secure said dome and said flange in a plurality of positions to a variety of human joints.

2. The joint pad and wrap of claim 1 further including at least one dome periphery adjustment assembly to shrink or expand the circumference of said dome to accommodate a variety of human joints.

3. The joint pad and wrap of claim 1 wherein said dome periphery adjustment assembly comprises in combination:
A. a cutout disposed in the lower periphery of said dome;
B. a tab disposed and attached to the bottom periphery of said dome bridging said cutout; and
C. mating opposing VELCRO pads attached to said dome and said tab to shrink or expand that portion of the circumference of said dome.

4. The joint pad and wrap of claiml wherein said insertable unit comprises a hot compress pack.

5. The joint pad and wrap of claiml wherein said insertable unit comprises a cold compress pack.

6. The joint pad and wrap of claiml wherein said insertable unit comprises a gel pack that is neither heated nor cooled.

7. A method for treating a medical disease or injury of a joint using a therapeutic joint pad and wrap comprising the steps of:
A. providing a joint pad and wrap comprising:
i. a dome shaped receptacle member;
ii. a pocket disposed within said dome, said pocket arranged to hold an insertable unit designed for treatment of a medical disease or injury of said joint;
iii. a crescent shaped compression flange member attached to the lower periphery of said dome, the combination of said dome and said flange forming a structure similar to a baseball cap; and
iv. a plurality of flexible straps attached to said dome and said flange to affix and secure said dome and said flange in a plurality of positions to a variety of said joints;
B. placing an insertable unit designed for treatment of a medical disease or injury of a joint into said pocket; and
C., securing said joint pad and wrap to said joint utilizing said flexible straps.

8. The joint pad and wrap of claim 7 wherein said insertable unit comprises a hot compress pack.

9. The joint pad and wrap of claim 7 wherein said insertable unit comprises a cold compress pack.

10. The joint pad and wrap of claim 7 wherein said insertable unit comprises a gel pack that is neither heated nor cooled.

11. The method of claim 7 wherein said flange is positioned above said joint.

12. The method of claim 7 wherein said flange is positioned below said joint.

13. The method of claim 7 wherein said joint comprises a knee.

14. The method of claim 7 wherein said joint comprises a heel.

15. The method of claim 7 wherein said joint comprises an elbow.

16. The method of claim 7 wherein said joint comprises a shoulder.
